(19) United States
(12) Patent Application Publication
(10) Pub. No.: US 2005/0004014 A1
(43) Pub. Date: Jan. 6, 2005

(54) New compound

(75) Inventors: Takahiro Matsuya, Osaka-shi (JP); Hiroaki Mizuno, Osaka-shi (JP); Takashi Tojo, Osaka-shi (JP); Hiroshi Matsuda, Osaka-shi (JP); David Barrett, Osaka-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: FUJISAWA PHARMACEUTICAL CO., LTD., Osaka-shi (JP)

(21) Appl. No.: 10/860,508

(22) Filed: Jun. 4, 2004

(30) Foreign Application Priority Data
Jun. 23, 2003 (AU) 2003903205

(51) Int. Cl. A61K 38/12; C07K 7/64

(52) U.S. Cl. 514/9; 530/317

(57) ABSTRACT

This invention relates to new lipopeptide compound represented by the following general formula (I): 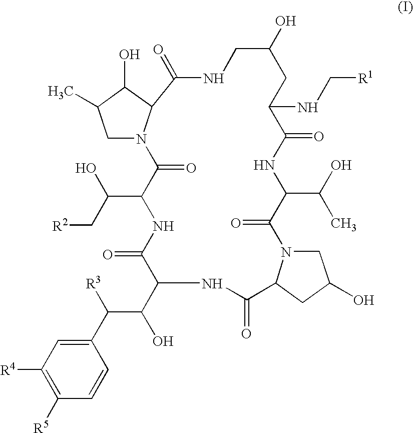 wherein
R¹, R², R³, R⁴ and R⁵ are as defined in the description or a salt thereof which has antimicrobial activities (especially, antifungal activities), inhibitory activity on β-1,3-glucan synthase, to process for preparation thereof, to a pharmaceutical composition comprising the same, and to a method for prophylactic and/or therapeutic treatment of infectious diseases including Pneumocystis carinii infection (e.g. Pneumocystis carinii pneumonia) in a human being or an animal.

### TECHNICAL FIELD

The present invention relates to new lipopeptide compounds and salts thereof which are useful as a medicament.

### BACKGROUND ART

In U.S. Pat. Nos. 5,376,634, 5,569,646, WO 96/11210, WO 99/40108, WO 00/64927 and WO 01/60846, there are disclosed the lipopeptide compound and a pharmaceutically acceptable salt thereof, which have antimicrobial activities (especially antifungal activity).

### DISCLOSURE OF INVENTION

The present invention relates to new lipopeptide compound and a salt thereof.

More particularly, it relates to new lipopeptide compound and a salt thereof, which have antimicrobial activities [especially, antifungal activities, in which the fungi may include Aspergillus, Cryptococcus, Candida, Mucor, Actinomyces, Histoplasma, Dermatophyte, Malassezia, Fusarium and the like.], inhibitory activity on β-1,3-glucan synthase, and further which are expected to be useful for the prophylactic and/or therapeutic treatment of Pneumocystis carinii infection (e.g. Pneumocystis carinii pneumonia) in a human being or an animal, to a process for preparation thereof, to a pharmaceutical composition comprising the same, and to a method for the prophylactic and/or therapeutic treatment of infectious disease including Pneumocystis carinii infection (e.g. Pneumocystis carinii pneumonia) in a human being or an animal.

The object lipopeptide compounds of the present invention are new and can be represented by the following general formula
(I): 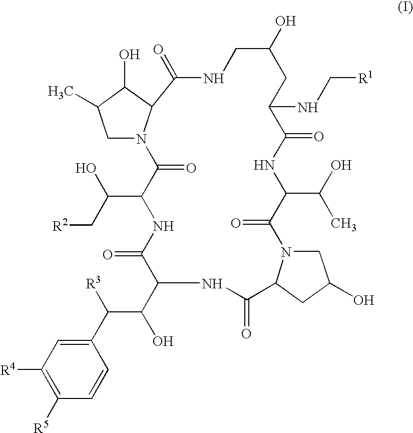
wherein
R¹ is aryl substituted with one or more suitable substituent (s),
R² is carbamoyl or amino(lower)alkyl which may be substituted with lower alkyl substituted with one or more hydroxy,
R³ is hydrogen or hydroxy,
R⁴ is hydrogen, hydroxy, lower alkoxy or amino(lower)alkoxy, and
R⁵ is hydroxy or acyloxy, or a salt thereof.

The new lipopeptide compound (I) or a salt thereof can be prepared by the process as illustrated in the following reaction schemes. 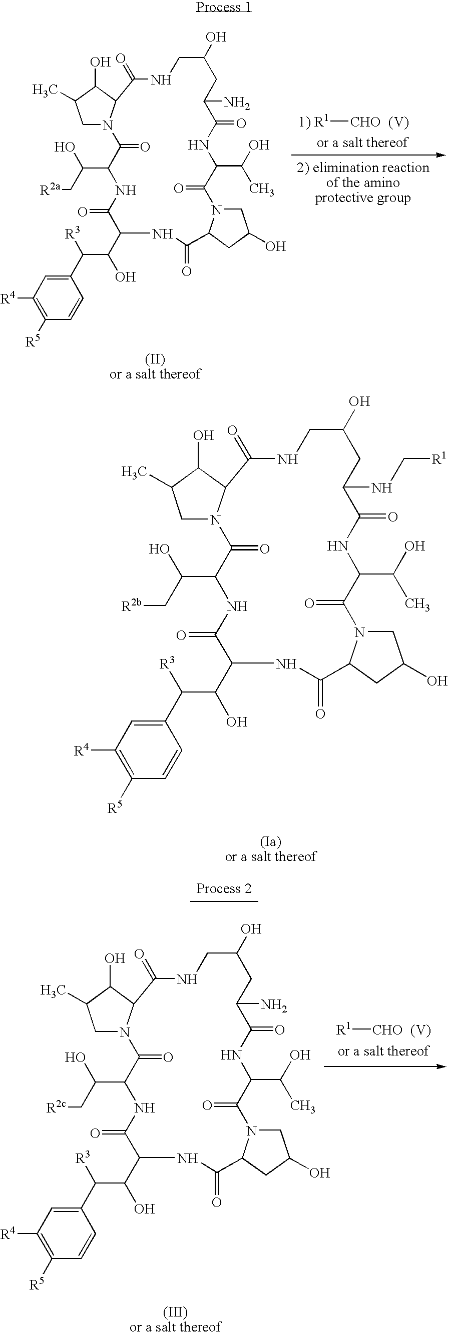 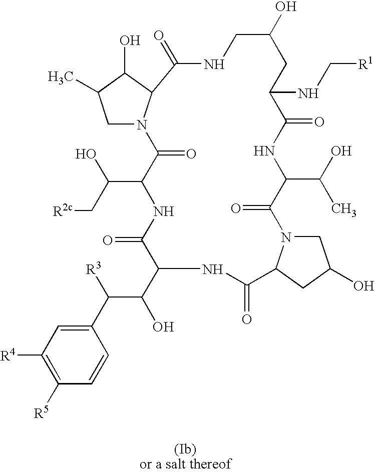
wherein R¹, R³, R⁴ and R⁵ are defined above,
R^{2a} is protected amino(lower)alkyl which may be substituted with lower alkyl substituted with one or more hydroxy,
R^{2b} is amino(lower)alkyl which may be substituted with lower alkyl substituted with one or more hydroxy, or
R^{2c} is carbamoyl.

Suitable salt of the new lipopeptide compound (I) is a pharmaceutically acceptable and conventional non-toxic salt, and may include a salt with a base or an acid addition salt such as a salt with an inorganic base, for example, an alkali metal salt (e.g., sodium salt, potassium salt, etc.), an alkaline earth metal salt (e.g., calcium salt, magnesium salt, etc.), an ammonium salt;
a salt with an organic base, for example, an organic amine salt (e.g., triethylamine salt, diisopropylethylamine salt, pyridine salt, picoline salt, ethanolamine salt, triethanolamine salt, dicyclohexylamine salt,
N,N′-dibenzylethylenediamine salt, 4-dimethylaminopyridine salt, etc.);
an inorganic acid addition salt (e.g., hydrochloride hydrobromide, sulfate, phosphate, etc.);
an organic carboxylic sulfonic acid addition salt (e.g., formate, acetate, trifluoroacetate, maleate, tartrate, fumarate, methanesulfonate, benzenesulfonate, toluenesulfonate, etc.);
a salt with a basic or acidic amino acid (e.g., arginine, aspartic acid, glutamic acid, etc.).

Suitable examples and illustration of the various definitions in the above and subsequent descriptions of the present specification, which the present invention intends to include within the scope thereof, are explained in detail as follows:

The term “lower” is used to intend a group having 1 to 6 carbon atom(s), unless otherwise provided.

Suitable example of “one or more” may be the number of 1 to 6, in which the preferred one may be the number of 1 to 3, and the most preferred one may be the number of 1 or 2.

Suitable example of “halogen” may be fluorine, chlorine, bromine, iodine and the like.

Suitable example of “lower alkoxy” may include straight or branched one such as methoxy, ethoxy, propoxy, isopropoxy, butoxy, isobutoxy, tert-butoxy, pentyloxy, tert-pentyloxy, neo-pentyloxy, hexyloxy, isohexyloxy and the like.

Suitable example of “higher alkoxy” may include straight or branched one such as heptyloxy, octyloxy, 3, 5-dimethyloctyloxy, 3, 7-dimethyloctyloxy, nonyloxy, decyloxy, undecyloxy, dodecyloxy, tridecyloxy, tetradecyloxy, hexadecyloxy, heptadecyloxy, octadecyloxy, nonadecyloxy, icosyloxy, and the like.

Suitable example of “lower alkyl” may include straight or branched one having 1 to 6 carbon atom(s), such as methyl, ethyl, propyl, isopropyl, butyl, isobutyl, sec-butyl, tert-butyl, pentyl, tert-pentyl, neo-pentyl, hexyl, isohexyl and the like.

Suitable example of “higher alkyl” may include straight or branched one such as heptyl, octyl, 3,5-dimethyloctyl, 3,7-dimethyloctyl, nonyl, decyl, undecyl, dodecyl, tridecyl, tetradecyl, hexadecyl, heptadecyl, octadecyl, nonadecyl, icosyl, and the like.

Suitable example of “aryl” and “ar” moiety may include phenyl which may have lower alkyl (e.g., phenyl, mesityl, xylyl, tolyl, etc.), naphthyl, anthryl, indanyl, fluorenyl, and the like, and this “aryl” and “ar” moiety may have one or more halogen.

Suitable example of “aroyl” may include benzoyl, toluoyl, naphthoyl, anthrylcarbonyl, and the like.

Suitable example of “heterocyclic group” may include
unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), etc.;
saturated 3 to 8-membered (more preferably 5 or 6-membered) heteromonocyclic group containing 1 to 4 nitrogen atom(s), for example, pyrrolidinyl, imidazolidinyl, piperidyl, piperazinyl, azetidinyl, etc.;
unsaturated condensed heterocyclic group containing 1 to 4 nitrogen atom(s), for example, indolyl, isoindolyl, indolinyl, indolizinyl, benzimidazolyl, quinolyl, isoquinolyl, indazolyl, benzotriazolyl, etc.;
unsaturated 3 to 8-membered (more preferably 5 or 6-membered) heteromonocyclic group containing 1 or 2 oxygen atom(s) and 1 to 3 nitrogen atom(s), for example, oxazolyl, isoxazolyl, oxadiazolyl (e.g., 1,2,4-oxadiazolyl, 1,3,4-oxadiazolyl, 1,2,5-oxadiazolyl, etc.), etc.;
saturated 3 to 8-membered (more preferably 5 or 6-membered) heteromonocyclic group containing 1 or 2 oxygen atom(s) and 1 to 3 nitrogen atom(s), for example, morpholinyl, sydnonyl, morpholino, etc.;
unsaturated condensed heterocyclic group containing 1 or 2 oxygen atom(s) and 1 to 3 nitrogen atom(s), for example, benzoxazolyl, benzoxadiazolyl, etc.;
unsaturated 3 to 8-membered (more preferably 5 or 6-membered) heteromonocyclic group containing 1 or 2 sulfur atom(s) and 1 to 3 nitrogen atom(s), for example, thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.), dihydrothiazinyl, etc.;
saturated 3 to 8-membered (more preferably 5 or 6-membered) heteromonocyclic group containing 1 or 2 sulfur atom(s) and 1 to 3 nitrogen atom(s), for example thiazolidinyl, thiomorpholinyl, thiomorpholino, etc.;
unsaturated 3 to 8-membered (more preferably 5 or 6-membered) heteromonocyclic group containing 1 or 2 sulfur atom(s), for example, thienyl, dihydrodithiinyl, dihydrodithionyl, etc.;
unsaturated condensed heterocyclic group containing 1 or 2 sulfur atom(s) and 1 to 3 nitrogen atom(s), for example, benzothiazolyl, benzothiadiazolyl, imidazothiadiazolyl, etc.;
unsaturated 3 to 8-membered (more preferably 5 or 6-membered) heteromonocyclic group containing an oxygen atom, for example, furyl etc.;
saturated 3 to 8-membered (more preferably 5 or 6-membered) heteromonocyclic group containing 1 or 2 oxygen atom(s), for example, tetrahydrofuran, tetrahydropyran, dioxacyclopentane, dioxacyclohexane, etc.;
unsaturated 3 to 8-membered (more preferably 5 or 6-membered) heteromonocyclic group containing an oxygen atom and 1 or 2 sulfur atom(s), for example, dihydrooxathiinyl, etc.;
unsaturated condensed heterocyclic group containing 1 or 2 sulfur atom(s), for example benzothienyl, benzodithiinyl, etc.;
unsaturated condensed heterocyclic group containing an oxygen atom and 1 or 2 sulfur atom(s), for example, benzoxathiinyl, etc.; and the like, and this “heterocyclic group” may have one or more suitable substituent(s) selected from the group consisting of lower alkyl, oxo, cyclo(lower)alkyl, hydroxy(lower)alkyl, carboxy(lower)alkanoyl which may have amino and heterocycliccarbonyl.

Suitable example of “cyclo(lower)alkyl” may include cyclopropyl, cyclobutyl, cyclopentyl, cyclohexyl, and the like, and this “cyclo(lower)alkyl” may have one or more lower alkyl.

Suitable example of “cyclo(lower)alkyloxy” may include cyclopropyloxy, cyclobutyloxy, cyclopentyloxy, cyclohexyloxy, and the like.

Suitable example of “acyl group” may include aliphatic acyl, aromatic acyl, arylaliphatic acyl and heterocyclic-aliphatic acyl derived from carboxylic acid, carbonic acid, carbamic acid, sulfonic acid, and the like.

Suitable example of said “acyl group” may be illustrated as follows.

Carboxy; carbamoyl; mono or di(lower)alkylcarbamoyl (e.g., methylcarbamoyl, dimethylcarbamoyl, ethylcarbamoyl, diethylcarbamoyl, etc.)

Aliphatic acyl such as lower or higher alkanoyl (e.g., formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, etc.);
lower or higher alkoxycarbonyl (e.g., methoxycarbonyl, ethoxycarbonyl, t-butoxycarbonyl, t-pentyloxycarbonyl, heptyloxycarbonyl, etc.); lower alkenyloxycarbonyl (e.g., vinyloxycarbonyl, propenyloxycarbonyl, allyloxycarbonyl, butenyloxycarbonyl, butedienyloxycarbonyl, pentenyloxycarbonyl, hexenyloxycarbonyl, etc.);
lower or higher alkylsulfonyl (e.g., methylsulfonyl, ethylsulfonyl, etc.);
lower or higher alkoxysulfonyl (e.g., methoxysulfonyl, ethoxysulfonyl, etc.); or the like;

Aromatic acyl such as aroyl (e.g., benzoyl, toluoyl, naphthoyl, etc.); ar(lower)alkanoyl [e.g., phenyl(C₁-C₆)alkanoyl (e.g., phenylacetyl, phenylpropanoyl, phenylbutanoyl, phenylisobutanoyl, phenylpentanoyl, phenylhexanoyl, etc.), naphthyl(C₁-C₆)alkanoyl (e.g., naphthylacetyl, naphthylpropanoyl, naphthylbutanoyl, etc.), etc.];
ar(lower)alkenoyl [e.g., phenyl(C₃-C₆)alkenoyl (e.g., phenylpropenoyl, phenylbutenoyl, phenylmethacryloyl, phenylpentanoyl, phenylhexenoyl, etc.), naphthyl(C₃-C₆)alkenoyl (e.g., naphthylpropenoyl, naphthylbutenoyl, etc.), etc.];
ar(lower)alkoxycarbonyl [e.g., phenyl(C₁-C₆)alkoxycarbonyl (e.g., benzyloxycarbonyl, etc.), fluorenyl(C₁-C₆)alkoxy-carbonyl (e.g., fluorenylmethyloxycarbonyl, etc.), etc.];
aryloxycarbonyl (e.g., phenoxycarbonyl, naphthyloxycarbonyl, etc.);
aryloxy(lower)alkanoyl (e.g., phenoxyacetyl, phenoxypropionyl, etc.);
arylcarbamoyl (e.g., phenylcarbamoyl, etc.);
arylthiocarbamoyl (e.g., phenylthiocarbamoyl, etc.);
arylglyoxyloyl (e.g., phenylglyoxyloyl, naphthylglyoxyloyl, etc.);
arylsulfonyl which may have 1 to 4 lower alkyl (e.g., phenylsulfonyl, p-tolylsulfonyl, etc.);
aroyl (e.g., benzoyl) substituted with one or more suitable substituent(s); or the like;

Heterocyclic acyl such as
heterocycliccarbonyl;
heterocyclic(lower)alkanoyl (e.g., heterocyclicacetyl, heterocyclicpropanoyl, heterocyclicbutanoyl, heterocyclicpentanoyl, heterocyclichexanoyl, etc.);
heterocyclic(lower)alkenoyl (e.g., heterocyclicpropenoyl, heterocyclicbutenoyl, heterocyclicpentenoyl, heterocyclichexenoyl, etc.);
heterocyclicglyoxyloyl; or the like; in which suitable “heterocyclic” moiety in the terms “heterocycliccarbonyl”, “heterocyclic(lower)alkanoyl”, “heterocyclic(lower)alkenoyl” and “heterocyclicglyoxyloyl” can be referred to aforementioned “heterocyclic” moiety.

Suitable example of “suitable substituent(s)” in the term of “aryl substituted with one or more suitable substituent(s)” may be
(1) heterocyclic group which may be substituted with aryl which may be substituted with optionally substituted heterocyclic group or
(2) aryl which may be substituted with heterocyclic group which may be substituted with optionally substituted cyclo(lower)alkyl,
in which preferred one may be
(1) heterocyclic group selected from the group consisting of thiadiazolyl, thiazolyl, piperazinyl and piperidyl, each of which may be substituted with phenyl which may be substituted with heterocyclic group selected from the group consisting of thiadiazolyl, thiazolyl, piperazinyl and piperidyl, each of which may be substituted with one or two substituent (s) selected from the group consisting of optionally substituted cyclo(lower)alkyl, lower alkoxy and lower alkyl or
(2) phenyl which may be substituted with heterocyclic group selected from the group consisting of thiadiazolyl, thiazolyl, piperazinyl and piperidyl, each of which may be substituted with cyclo(lower)alkyl which may be substituted with one or two substituent (s) selected from the group consisting of optionally substituted cyclo(lower)alkyl, lower alkoxy and lower alkyl, and
the more preferred one may be
(1) thiadiazolyl substituted with phenyl substituted with piperazinyl substituted with cyclo(lower)alkyl which may be substituted with one or two lower alkyl,
(2) thiadiazolyl substituted with phenyl substituted with piperidyl substituted with one or two substituent(s) selected from the group consisting of cyclo(lower)alkyl, lower alkyl and lower alkoxy,
(3) phenyl substituted with piperazinyl substituted with cyclo(lower)alkyl substituted with cyclo(lower)alkyl and lower alkoxy or
(4) thiazolyl substituted with phenyl substituted with piperidyl substituted with one or two substituent(s) selected from the group consisting of cyclo(lower)alkyl, lower alkyl and lower alkoxy, and
the most preferred one may be
(1) thiadiazolyl substituted with phenyl substituted with piperazinyl substituted with cyclohexyl which may be substituted with methyl,
(2) thiadiazolyl substituted with phenyl substituted with piperidyl substituted with one or two substituent(s) selected from the group consisting of cyclohexyl, butyl and methoxy,
(3) phenyl substituted with piperazinyl substituted with cyclohexyl substituted with methoxy and cyclohexyl or
(4) thiazolyl substituted with phenyl substituted with piperidyl substituted with one or two substituent(s) selected from the group consisting of cyclohexyl, butyl and methoxy.

The more suitable example of “aryl substituted with one or more suitable substituent(s)” may be
(1) phenyl substituted with thiadiazolyl substituted with phenyl substituted with piperazinyl substituted with cyclohexyl which may be substituted with methyl,
(2) phenyl substituted with thiadiazolyl substituted with phenyl substituted with piperidyl substituted with cyclohexyl and methoxy,
(3) phenyl substituted with thiadiazolyl substituted with phenyl substituted with piperidyl substituted with butyl and methoxy,
(4) phenyl substituted with phenyl substituted with piperazinyl substituted with cyclohexyl substituted with methoxy and cyclohexyl or
(5) phenyl substituted with thiazolyl substituted with phenyl substituted with piperidyl substituted with butyl and methoxy.

Suitable example of “lower alkyl” in the term of “lower alkyl substituted with one or more hydroxy” can be referred to aforementioned “lower alkyl”, in which the preferred one may be methyl, ethyl, propyl, isopropyl, butyl, pentyl and hexyl.

Suitable example of “lower alkyl substituted with one or more hydroxy” may be dihydroxypropyl, dihydroxyisopropyl, trihydroxybutyl, tetrahydroxypentyl, pentahydroxyhexyl and diacetyloxyisopropyl.

Suitable example of “amino protective group” may be included in aforementioned “acyl group”, in which the preferred one may be ar(lower)alkoxycarbonyl and lower alkoxycarbonyl, and the most preferred one may be acetyl, 2-acetyloxypropionyl, methylsulfonyl, 2,5-diaminopentanoyl, benzyloxycarbonyl, fluorenylmethoxycarbonyl, allyloxycarbonyl and tert-butoxycarbonyl.

Suitable example of “acyl” moiety of “acyloxy” can be referred to aforementioned “acyl group”, in which the preferred one may be lower alkenyloxycarbonyl, and the most preferred one may be allyloxycarbonyl.

Suitable example of “acyloxy” may be lower alkenyloxycarbonyloxy, and the more preferred one may be allyloxycarbonyloxy.

Suitable example of “lower alkyl” in the term of “amino(lower)alkyl” can be referred to aforementioned “lower alkyl”, in which the preferred one may be (C₁- C₃)alkyl, and the most preferred one may be methyl and ethyl.

Particularly, the preferred examples of the cyclic lipopeptide compound (I) of the present invention are as follows:
the compound (I), wherein
R¹ is (1) phenyl substituted with thiadiazolyl substituted with phenyl substituted with piperazinyl substituted with cyclo(lower)alkyl which may be substituted with one or two lower alkyl,
(2) phenyl substituted with thiadiazolyl substituted with phenyl substituted with piperidyl substituted with one or two substituent(s) selected from the group consisting of cyclo(lower)alkyl, lower alkyl and lower alkoxy,
(3) phenyl substituted with phenyl substituted with piperazinyl substituted with cyclo(lower)alkyl substituted with cyclo(lower)alkyl and lower alkoxy or
(4) phenyl substituted with thiazolyl substituted with phenyl substituted with piperidyl substituted with one or two substituent(s) selected from the group consisting of cyclo(lower)alkyl, lower alkyl and lower alkoxy
R² is carbamoyl or amino(lower)alkyl which may be substituted with lower alkyl substituted with two hydroxy,
R³ is hydrogen,
R⁴ is hydrogen, hydroxy, lower alkoxy or amino (lower)alkoxy, and
R⁵ is hydroxy.

And, more preferred one may be the compound (I)
Wherein
R¹ is (1) phenyl substituted with thiadiazolyl substituted with phenyl substituted with piperazinyl substituted with cyclohexyl which may be substituted with methyl,
(2) phenyl substituted with thiadiazolyl substituted with phenyl substituted with piperidyl substituted with cyclohexyl and methoxy,
(3) phenyl substituted with thiadiazolyl substituted with phenyl substituted with piperidyl substituted with butyl and methoxy,
(4) phenyl substituted with phenyl substituted with piperazinyl substituted with cyclohexyl substituted with methoxy and cyclohexyl or
(5) phenyl substituted with thiazolyl substituted with phenyl substituted with piperidyl substituted with butyl and methoxy,
R² is amino(lower)alkyl substituted with lower alkyl substituted with two hydroxy,
R³ is hydrogen,
R⁴ is lower alkoxy, and
R⁵ is hydroxy.

The processes for preparing the lipopeptide compound (I) of the present invention are explained in detail in the following.

Process 1

1) The object compound (Ia) or a salt thereof can be prepared by subjecting the compound (II) or a salt thereof with the compound (V) of the formula:
R¹—CHO (V)
or a salt thereof to the condensing reaction, and then, to the elimination reaction of the amino protective group.

The condensing reaction is carried out in a conventional manner, including chemical reduction and catalytic reduction.

Suitable reducing agents to be used in chemical reduction are hydrides [e.g., hydrogen iodide, hydrogen sulfide, lithium aluminum hydride, sodium borohydride, sodium cyanoborohydride, etc.], or a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium, sulfate, palladium on barium carbonate, etc.], nickel catalysts [e.g. reduced nickel, nickel oxide, Raney nickel, etc.], cobalt catalysts [e.g. reduced cobalt, Raney cobalt, etc], iron catalysts [e.g. reduced iron, Raney iron, etc], copper catalysts [e.g. reduced copper, Raney copper, Ullman copper, etc.] and the like.

The reduction is usually carried out in a conventional solvent which does not adversely influence the reaction such as water, methanol, ethanol, propanol, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to warming.

2) The elimination reaction of the amino protective group is carried out in accordance with a conventional method such as hydrolysis, reduction or the like.

The hydrolysis is preferably carried out in the presence of a base or an acid including Lewis acid. Suitable base may include an inorganic base and an organic base such as an alkali metal [e.g. sodium, potassium, etc.], an alkaline earth metal [e.g. magnesium, calcium, etc.], the hydroxide or carbonate or bicarbonate thereof, trialkylamine [e.g. trimethylamine, triethylamine, etc.], picoline, 1,5-diazabicyclo[4.3.O]non-5-ene, 1,4-diazabicyclo[2.2.2]octane, 1,8-diazabicyclo[5.4.0]undec-7-ene, or the like.

Suitable acid may include an organic acid [e.g. formic acid, acetic acid, propionic acid, trichloroacetic acid, trifluoroacetic acid, etc.] and an inorganic acid [e.g. hydrochloric acid, hydrobromic acid, sulfuric acid, hydrogen chloride, hydrogen bromide, etc.]. The elimination using Lewis acid such as trihaloacetic acid [e.g. trichloroacetic acid, trifluoroacetic acid, etc.] or the like is preferably carried out in the presence of cation trapping agents [e.g. anisole, phenol, etc.]

The reaction is usually carried out in a solvent such as water, an alcohol [e.g. methanol, ethanol, etc.], methylene chloride, tetrahydrofuran, a mixture thereof or any other solvent which does not adversely influence the reaction. A liquid base or acid can be also used as the solvent. The reaction temperature is not critical and the reaction is usually carried out under cooling to warming.

The reduction method applicable for the elimination reaction may include chemical reduction and catalytic reduction.

Suitable reducing agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium, sulfate, palladium on barium carbonate, etc.], nickel catalysts [e.g. reduced nickel, nickel oxide, Raney nickel, etc.], cobalt catalysts [e.g. reduced cobalt, Raney cobalt, etc], iron catalysts [e.g. reduced iron, Raney iron, etc], copper catalysts [e.g. reduced copper, Raney copper, Ullman copper, etc.] and the like.

The reduction is usually carried out in a conventional solvent which does not adversely influence the reaction such as water, methanol, ethanol, propanol, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to warming.

Process 2

The object compound (Ib) or a salt thereof can be prepared by subjecting the compound (III) or a salt thereof with the compound (V) of the formula:
R¹—CHO (V)
or a salt thereof to the condensing reaction.

The condensing reaction is carried out in a conventional manner, including chemical reduction and catalytic reduction.

Suitable reducing agents to be used in chemical reduction are hydrides [e.g., hydrogen iodide, hydrogen sulfide, lithium aluminum hydride, sodium borohydride, sodium cyanoborohydride, etc.], or a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium, sulfate, palladium on barium carbonate, etc.], nickel catalysts [e.g. reduced nickel, nickel oxide, Raney nickel, etc.], cobalt catalysts [e.g. reduced cobalt, Raney cobalt, etc], iron catalysts [e.g. reduced iron, Raney iron, etc], copper catalysts [e.g. reduced copper, Raney copper, Ullman copper, etc.] and the like.

The reduction is usually carried out in a conventional solvent which does not adversely influence the reaction such as water, methanol, ethanol, propanol, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to warming.

The compounds obtained by the above Processes 1 and 2 can be isolated and purified by a conventional method such as pulverization, recrystallization, column-chromatography, high-performance liquid chromatography (HPLC), reprecipitation, desalting resin column chromatography, or the like.

The compounds obtained by the above Processes 1 and 2 may be obtained as its solvate (e.g., hydrate, ethanolate, etc.), and its solvate (e.g., hydrate, ethanolate, etc.) is included within the scope of the present invention.

It is to be noted that each of the lipopeptide compound (I) may include one or more stereoisomer such as optical isomer(s) and geometrical isomer(s) due to asymmetric carbon atom(s) and double bond(s) and all such isomers and the mixture thereof are included within the scope of the present invention.

The-lipopeptide compound (I) or a salt thereof may include solvated compound [e.g., hydrate, ethanolate, etc.].

The lipopeptide compound (I) or a salt thereof may include both its crystal form and non-crystal form.

It should be understood that the lipopeptide compound (I) of the present invention may include the prodrug form.

The patent applications and publications cited herein are incorporated by reference.

In order to show the usefulness of the lipopeptide compound (I) of the present invention, the biological data of the representative compound is explained in the following.

### Biological Property of the Lipopeptide

### Compound (I) of the Present Invention

Test (Antimicrobial activity):

In vitro antimicrobial activity of the object compound of Examples 1, 2, 3 and 4 disclosed later was determined by MIC_{S} in mouse serum as described below.

Test Method:

The MIC_{S} in mouse serum were determined by the microdilution method using ICR mouse serum buffered with 20 mM HEPES buffer (pH 7.3) as a test medium. Inoculum suspension of 10⁶ cells/ml were prepared by a hemocytometric procedure and diluted to obtain an inoculum size of approximately 1.0×10³ cells/ml. Microplates were incubated at 37° C. for 24 hours in 5% CO₂. The MIC_{S} were defined as the lowest concentrations at which no visible growth was observed.

Test Result:

MIC (μg/ml)

| | Test organism |
|---|---|
| Test compound | Candida albicans FP-633 |
| The object compound of | <0.15 |
| Example 1 | |
| The object compound of | <0.15 |
| Example 2 | |
| The object compound of | <0.15 |
| Example 3 | |
| The object compound of | <0.15 |
| Example 4 | |

From the test result, it is realized that the lipopeptide compound (I) of the present invention has an antimicrobial activity (especially, antifungal activity).

In more details, the lipopeptide compound (I) of the present invention have an antifungal activity, particularly against the following fungi.
Acremonium;
Absidia (e.g., Absidia corymbifera, etc);
Aspergillus (e.g., Aspergillus clavatus, Aspergillus flavus, Aspergillus fumigatus, Aspergillus nidulans, Aspergillus niger, Aspergillus terreus, Aspergillus versicolor, etc); Blastomyces (e.g., Blastomyces dermatitidis, etc);
Candida (e.g., Candida albicans, Candida glabrata, Candida guilliermondii, Candida kefyr, Candida krusei, Candida parapsilosis, Candida stellatoidea, Candida tropicalis, candida utilis, etc.);
Cladosporium (e.g., Cladosporium trichloides, etc);
Coccidioides (e.g., Coccidioides immitis, etc);
Cryptococcus (e.g., Cryptococcus neoformans, etc);
Cunninghamella (e.g., Cunninghamella elegans, etc);
Dermatophyte;
Exophiala (e.g., Exophiala dermatitidis, Exophiala spinifera, etc);
Epidermophyton (e.g., Epidermophyton floccosum, etc);
Fonsecaea (e.g., Fonsecaea pedrosoi, etc);
Fusarium (e.g., Fusarium solani, etc);
Geotrichum (e.g., Geotrichum candiddum, etc);
Histoplasma (e.g., Histoplasma capsulatum var. capsulatum, etc).
Malassezia (e.g., Malassezia furfur, etc);
Microsporum (e.g., Microsporum canis, Microsporum gypseum, etc);
Mucor;
Paracoccidioides (e.g., Paracoccidioides brasiliensis, etc);
Penicillium (e.g., Penicillium marneffei, etc);
Phialophora;
Pneumocystis (e.g., Pneumocystis carinii, etc);
Pseudallescheria (e.g., Pseudallescheria boydii, etc);
Rhizopus (e.g., Rhizopus microsporus var. rhizopodiformis, Rhizopus oryzae, etc);
Saccharomyces (e.g., Saccharomyces cerevisiae, etc);
Scopulariopsis;
Sporothrix (e.g., Sporothrix schenckii, etc);
Trichophyton (e.g., Trichophyton mentagrophytes, Trichophyton rubrum, etc);
Trichosporon (e.g., Trichosporon asahii, Trichosporon cutaneum, etc).

The above fungi are well-known to cause various infection diseases in skin, eye, hair, nail, oral mucosa, gastrointestinal tract, bronchus, lung, endocardium, brain, meninges, urinary organ, vaginal protion, oral cavity, ophthalmus, systemic, kidney, bronchus, heart, external auditory canal, bone, nasal cavity, paranasal cavity, spleen, liver, hypodermal tissue, lymph doct, gastrointestine, articulation, muscle, tendon, interstitial plasma cell in lung, blood, and so on.

Therefore, the lipopeptide compound (I) of the present invention are useful for preventing and treating various infectious diseases, such as dermatophytosis (e.g., trichophytosis, etc), pityriasis versicolor, candidiasis, cryptococcosis, geotrichosis, trichosporosis, aspergillosis, penicilliosis, fusariosis, zygomycosis, sporotrichosis, chromomycosis, coccidioidomycosis, histoplasmosis, blastomycosis, paracoccidioidomycosis, pseudallescheriosis, mycetoma, mycotic keratitis, otomycosis, pneumocystosis, fungemia, and so on.

The combination use of azoles such as fluconazole, voriconazole, itraconazole, ketoconazole, miconazole, ravuconazole and posaconazole; polyenes such as amphotericin B, nystatin, liposamal and lipid forms thereof such as Abelcet, AmBisome, and Amphocil; purine or pyrimidine nucleotide inhibitors such as flucytosine; or polyxins such as nikkomycines, in particular nikkomycine Z or nikkomycine X; other chitin inhibitors; elongation factor inhibitors such as sordarin and analogs thereof; mannan inhibitors such as predamycin, bactericidal/permeability-inducing (BPI) protein products such as XMP.97 or XMP.127; or complex carbohydrate antifungal agents such as CAN-296 with the lipopeptide compound (I) or salt thereof is effective against above diseases.

The combination use of immunosuppressant such as tacrolimus, or G-CSF (Granulocyte-colony stimulating factor) with the lipopeptide compound (I) or a salt thereof is effective against above infectious diseases.

The pharmaceutical composition of the present invention can be used in the form of a pharmaceutical preparation, for example, in solid, semisolid or liquid form, which contains the lipopeptide compound (I) or a pharmaceutically acceptable salt thereof, as an active ingredient in admixture with an organic or inorganic carrier or excipient which is suitable for rectal; pulmonary (nasal or buccal inhalation); ocular; external (topical); oral administration; parenteral (including subcutaneous, intravenous and intramuscular) administrations; insufflation (including aerosols from metered dose inhalator); nebulizer; or dry powder inhalator.

The active ingredient may be compounded, for example, with the usual non-toxic, pharmaceutically acceptable carriers in a solid form such as granules, tablets, dragees, pellets, troches, capsules, or suppositories; creams; ointments; aerosols; powders for insufflation; in a liquid form such as solutions, emulsions, or suspensions for injection; ingestion; eye drops; and any other form suitable for use. And, if necessary, there may be included in the above preparation auxiliary substance such as stabilizing, thickening, wetting, emulsifying and coloring agents; perfumes or buffer; or any other commonly may be used as additives.

The lipopeptide compound (I) or a pharmaceutically acceptable salt thereof is/are included in the pharmaceutical composition in an amount sufficient to produce the desired antimicrobial effect upon the process or condition of diseases.

For applying the composition to humans, it is preferable to apply it by intravenous, intramuscular, pulmonary, oral administration, eye drop administration or insufflation. While the dosage of therapeutically effective amount of the lipopeptide compound (I) varies from and also depends upon the age and condition of each individual patient to be treated, in the case of intravenous administration, a daily dose of 0.01-400 mg of the lipopeptide compound (I) per kg weight of human being in the case of intramuscular administration, a daily dose of 0.1-20 mg of the lipopeptide compound (I) per kg weight of human being, in case of oral administration, a daily dose of 0.5-50 mg of the lipopeptide compound (I) per kg weight of human being is generally given for treating or preventing infectious diseases.

Especially in case of the treatment of prevention of Pneumocystis carinii infection, the followings are to be noted.

For administration by inhalation, the compounds of the present invention are conveniently delivered in the form of an aerosol spray presentation form pressurized as powders which may be formulated and the powder compositions may be inhaled with the aid of an insufflation powder inhaler device. The preferred delivery system for inhalation is a metered dose inhalation aerosol, which may be formulated as a suspension or solution of compound in suitable propellants such as fluorocarbons or hydrocarbons.

Because of desirability to directly treat lung and bronchi, aerosol administration is a preferred method of administration. Insufflation is also a desirable method, especially where infection may have spread to ears and other body cavities.

Alternatively, parenteral administration may be employed using drip intravenous administration.

For administration by intravenous administration, the preferred pharmaceutical composition is the lyophilized form containing the lipopeptide compound (I) or its pharmaceutically acceptable salt.

The amount of the lipopeptide compound (I) or its pharmaceutically acceptable salt contained in the composition for a single unit dosage of the present invention is 0.1 to 400 mg, more preferably 1 to 200 mg, still more preferably 5 to 100 mg, specifically 5, 10, 15, 20, 25, 30, 35, 40, 45, 50, 55, 60, 70, 75, 80, 85, 90, 95 and 100 mg.

The present invention further provides the following ones.

An article of manufacture, comprising packaging material and the compound (I) identified in the above contained within said packaging material, wherein said the compound (I) is therapeutically effective for preventing or treating infectious diseases caused by pathogenic microorganism, and wherein said packaging material comprises a label or a written material which indicates that said compound (I) can or should be used for preventing or treating infectious diseases caused by pathogenic microorganism.

A commercial package comprising the pharmaceutical composition containing the compound (I) identified in the above and a written matter associated therewith, wherein the written matter states that the compound (I) can or should be used for preventing or treating infectious diseases caused by pathogenic microorganism.

The following Preparations and Examples are given for the purpose of illustrating the present invention in more detail.

Preparation 1

A mixture of 4-[5-[4-(4-butyl-4-methoxy-1-piperidyl)phenyl]-1,3,4-thiadiazol-2-yl]phenylmethanol (1.17 g) and minganese dioxide (2.32 g) in chloroform (234 ml) was stirred for 54.5 hours at ambient temperature. The mixture was filtered by celite and the filtrate was concentrated under reduced pressure to give 4-[5-[4-(4-butyl-4-methoxy-1-piperidyl)phenyl]-1,3,4-thiadiazol-2-yl]benzaldehyde (973 mg)

NMR (CDCl₃, σ):0.93 (3H, t, J=6.4 Hz), 1.2-2.0 (10H, m), 3.1-3.3 (5H, m), 3.5-3.7 (2H, m), 6.97 (2H, d, J=8.9 Hz), 7.89 (2H, d, J=8.9 Hz), 8.00 (2H, d, J=8.3 Hz), 8.17 (2H, d, J=8.3 Hz), 10.08 (1H, s) MASS (ESI+):m/z 436.07 (M+H)

Preparation 2

To a solution of methyl 4-[5-[4-(4-butyl-4-methoxy-1-piperidyl)phenyl]-1,3,4-thiadiazol-2-yl]benzoate (1.38 g) in tetrahydrofuran (41.4 ml) was added lithium aluminum hydride (169 mg) and stirred for 1.5 hour at room temperature. To the reaction mixture was added ethyl acetate (1.74 ml) and stirred for a half hour at room temperature. To the reaction mixture was added water. And the reaction mixture was adjusted to pH 3 with 1N HCl and the resulting precipitate was collected by filtration, washed with water and acetonitrile, then dried to give 4-[5-[4-(4-butyl-4-methoxy-1-piperidyl)phenyl]-1,3,4-thiadiazol-2-yl]phenylmethanol (1.176 g).

NMR (CDCl₃, σ):0.93 (3H, t, J=6.3 Hz), 1.2-2.0 (11H, m), 3.05-3.3 (5H, m), 3.45-3.65 (2H, m), 4.78 (2H, s), 6.96 (2H, d, J=8.9 Hz), 7.48 (2H, d, J=8.2 Hz), 7.87 (2H, d, J=8.9 Hz), 7.98 (2H, d, J=8.2 Hz) MASS (ESI+):m/z 460.33 (M+Na)

Preparation 3

A suspension of methyl 4-[2-[4-(4-butyl-4-methoxy-1-piperidyl)benzoyl]-hydrazinocarbonyl]benzoate(1.5 g) and diphosphorus pentasulfide (1.07 g) in dimethoxyethane (45 ml) was stirred for 2 hours at 100° C. To the reaction mixture was added water. The reaction mixture was adjusted to pH 7.5 with 1N NaOHaq. And the resulting precipitate was collected by filtration, washed with water and acetonitrile, and then dried to give methyl 4-[5-[4-(4-butyl-4-methoxy-1-piperidyl)phenyl]-1,3,4-thiadiazol-2-yl]benzoate (1.39 g).

NMR (CDCl₃, σ):0.90 (3H, t, J=6.4 Hz), 1.2-2.5 (10H, m), 3.19 (3H, s), 3.2-3.35 (2H, m), 3.5-3.65 (2H, m), 3.96 (3H, s), 7.10 (2H, d, J=8.8 Hz), 7.96 (2H, d, J=8.8 Hz), 8.06 (2H, d, J=8.4 Hz), 8.15 (2H, d, J=8.4 Hz) MASS (ESI+):m/z 466.2 (M+H)

Preparation 4

To a solution of 4-(4-butyl-4-methoxy-1-piperidyl)benzohydrazide (2.53 g) in tetrahydrofuran (76 ml) and pyridine (2.01 ml) was added methyl 4-(chlorocarbonyl)benzoate (1.73 g) at 0° C. The reaction mixture was stirred for 6.5 hours at room temperature and poured into water. The mixture was adjusted to pH 9 with 1N NaOH aq. and the resulting precipitate was collected by filtration, washed with water, isopropanol and diisopropylether, and then dried to give methyl 4-[2-[4-(4-butyl-4-methoxy-1-piperidyl)benzoyl]hydrazinocarbonyl]benzoate (3.01 g).

NMR (CDCl₃, σ):0.92 (3H, t, J=6.4 Hz), 1.2-1.95 (10H, m), 3.05-3.3 (5H, m), 3.45-3.65 (2H, m), 3.95 (3H, s), 6.87 (2H, d, J=8.9 Hz), 7.75 (2H, d, J=8.9 Hz), 7.92 (2H, d, J=8.4 Hz), 8.10 (2H, d, J=8.4 Hz), 9.26 (1H, d, J=5.7 Hz), 9.75 (1H, d, J=5.7 Hz) MASS (ESI+):m/z 490.2 (M+Na)

Preparation 5

To a solution of ethyl 4-(4-butyl-4-methoxy-1-piperidyl)benzoate (2.85 g) in ethanol (56 ml) and tetrahydrofuran (23 ml) was added hydrazine monohydrate (39 ml) and the mixture was stirred for 7 hours at 100° C. After cooling, the solvent was removed under reduced pressure. Water was added and the precipitate was collected by filtration, washed with water and dried under reduced pressure to give 4-(4-butyl-4-methoxy-1-piperidyl)benzohydrazide (2.54 g).

NMR (CDCl₃, σ):0.92 (3H, t, J=6.4 Hz), 1.2-1.95 (10H, m), 3.05-3.25 (5H, m), 3.4-3.6 (2H, m), 4.05 (2H, brs), 6.8-6.95 (2H, m), 7.17 (1H, s), 7.55-7.7 (2H, m) MASS (ESI+):m/z 306.3 (M+H)

Preparation 6

To a solution of 4-butyl-4-methoxypiperidine trifluoroacetate (3.73 g) and ethyl 4-fluorobenzoate (1.75 ml) in dimethylsulfoxide (20 ml) was added potassium carbonate (4.93 g). The solution was stirred for 5 hours at 150° C. The reaction mixture was added to a mixture of water and ethyl acetate. The organic layer was washed with brine and dried over magnesium sulfate. The magnesium sulfate was filtered off, and the filtrate was concentrated under reduced pressure. The residue was purified by silica gel chromatography (10:1 hexane-ethyl acetate elution) to give ethyl 4-(4-butyl-4-methoxy-1-piperidyl)-benzoate (2.859 g).

NMR (CDCl₃, σ):0.92 (3H, t, J=6.4 Hz), 1.2-1.95 (13H, m), 3.05-3.3 (5H, m), 3.45-3.65 (2H, m), 4.32 (2H, q, J=7.1 Hz), 6.86 (2H, d, J=9.0 Hz), 7.90 (2H, d, J=9.0 Hz) MASS (ESI+):m/z 320.1 (M+H)

Preparation 7

To a solution of tert-butyl 4-butyl-4-methoxy-1-piperidinecarboxylate (8.82 g) and anisole (24.7 ml) in dichloromethane (44 ml) was added dropwise with stirring trifluoroaceticacid (50.1 ml) at 0° C. The mixture was stirred for a half hour at room temperature. The solvent was concentrated under reduced pressure to give 4-butyl-4-methoxypiperidine trifluoroacetate (13.778 g).

NMR (CDCl₃, σ):0.92 (3H, t, J=6.7 Hz), 1.15-2.05 (10H, m), 3.05-3.35 (7H, m), 8.0-8.6 (2H, m) MASS (ESI+):m/z 172.3 (M+H)

Preparation 8

To a solution of tert-butyl 4-butyl-4-hydroxy-1-piperidinecarboxylate (11.07 g) in N,N-dimethylformamide(110 ml) was added sodium hydride (60% dispersion in mineral oil) (1.55 g). The solution was stirred for 1.5 hour at 60° C. To the reaction mixture was added iodomethane (8.03 ml) . The mixture was stirred for 4 hours at room temperature. The reaction mixture was added to a mixture of water and ethyl acetate. The organic layer was washed with brine and dried over magnesium sulfate. The magnesium sulfate was filtered off, and the filtrate was concentrated under reduced pressure. The residue was purified by silica gel chromatography (10:1 hexane-ethyl acetate elution) to give tert-butyl 4-butyl-4-methoxy-1-piperidinecarboxylate (8.83 g).

NMR (CDCl₃, σ):0.91 (3H, t, J=6.5 Hz), 1.05-1.5 (17H, m), 1.65-1.8 (2H, m), 2.9-3.2 (5H, m), 3.6-3.9 (2H, m) MASS (ESI+):m/z 294.2 (M+Na)

Preparation 9

To the solution of n-butyl magnesium chloride (3.0 M solution in dimethyl ether)(33.7 ml) was added dropwise with stirring tert-butyl 4-oxo-1-piperidinecarboxylate(10 g) in tetrahydrofuran (50 ml) for 3 hours at 0° C. To the reaction mixture was added water. And the mixture was adjusted to pH 3 with 1N HCl. The organic layer was washed with brine and dried over magnesium sulfate. The magnesium sulfate was filtered off, and the filtrate was concentrated under reduced pressure. The residue was purified by silica gel chromatography (5:1-3:1 hexane-ethyl acetate elution) to give tert-butyl 4-butyl-4-hydroxy-1-piperidinecarboxylate (11.076 g).

NMR (CDCl₃, σ):0.92 (3H, t, J=6.8 Hz), 1.1-1.6 (20H, m), 3.0-3.3 (2H, m), 3.65-3.95 (2H, m) MASS (ESI+):m/z 280.3 (M+Na)

Preparation 10

A mixture of 1-[4-[5-[4-[4-(4-methylcyclohexyl)-1-piperazinyl]phenyl]-1,3,4-thiadiazol-2-yl]benzoyloxy]-1H-1,2,3-benzotriazole (5 g), N,O-dimethylhydroxylamine hydrochloride (925 mg) and diisopropylethylamine (2.25 ml) in N,N-dimethylformamide(100 ml) was stirred for 4 hours. And the reaction mixture was poured into water. The resulting precipitates were filtered, washed with water and acetonitrile and dried to give N-methoxy-N-methyl-4-[5-[4-[4-(cis-4-methylcyclohexyl)-1-piperazinyl]phenyl]-1,3,4-thiadiazol-2-yl]benzamide (3.845 g).

NMR (CDCl₃, σ):0.94 (3H, d, J=6.9 Hz), 1.3-2.4 (10H, m), 2.6-2.85 (4H, m), 3.25-3.45 (7H, m), 3.58 (3H, s), 6.96 (2H, d, J=8.8 Hz), 7.81 (2H, d, J=8.3 Hz), 7.90 (2H, d, J=8.8 Hz), 8.03 (2H, d, J=8.3 Hz) MASS (ESI+):m/z 506.2 (M+H)

The following compound was obtained in substantially the same manner as that of Preparation 10.

Preparation 11

### N-methoxy-4′-[4-[(1s,4s)-1-methoxy-1,1′-bi(cyclohexyl)-4-yl]-1-piperazinyl]-N-methyl-1,1′-biphenyl-4-carboxamide

NMR (CDCl₃, σ):0.7-1.9 (19H, m), 2.15-2.4 (1H, m), 2.7-2.85 (4H, m), 3.11 (3H, s), 3.2-3.35 (4H, m), 3.38 (3H, s), 3.60 (3H, s), 7.00 (2H, d, J=8.8 Hz), 7.45-7.65 (4H, m), 7.74 (2H, d, J=8.4 Hz) MASS (ESI+):m/z 520.5 (M+H)

The following compound was obtained in substantially the same manner as that of Preparation 10.

Preparation 12

### 4-[5-[4-(4-cyclohexyl-4-methoxy-1-piperidyl)phenyl]-1,3,4-thiadiazol-2-yl]-N-methoxy-N-methylbenzamide

NMR (CDCl₃, σ):1.7-1.9 (15H, m), 3.0-3.25 (5H, m), 3.40 (3H, s), 3.5-3.7 (5H, m), 6.97 (2H, d, J=8.9 Hz), 7.80 (2H, d, J=8.4 Hz), 7.88 (2H, d, J=8.9 Hz), 8.03 (2H, d, J=8.4 Hz) MASS (ESI+):m/z 521.2 (M+H)

Preparation 13

To a solution of 4-[5-[4-(4-cyclohexyl-1-piperazinyl)phenyl]-1,3,4-thiadiazol-2-yl]-N-methoxy-N-methylbenzamide (2.2 g) in tetrahydrofuran (44 ml) was added lithium aluminum hydride (170 mg) at 0° C. in stream of nitrogen. The mixture was then stirred for 1.5 hour. To the reaction mixture was added sodium fluoride (752 mg), water (0.242 ml) and chloroform. The resulting precipitates was filtered off, and the filtrate was concentrated under reduced pressure to give 4-[5-[4-(4-cyclohexyl-1-piperazinyl)phenyl]-1,3,4-thiadiazol-2-yl]benzaldehyde(1.9 mg)

NMR (CDCl₃, σ):1.1-2.0 (10H, m), 2.2-2.45 (1H, m), 2.74 (4H, t, J=5.1 Hz), 3.35 (4H, t, J=5.1 Hz), 6.97 (2H, d, J=8.9 Hz), 7.91 (2H, d, J=8.9 Hz), 8.00 (2H, d, J=8.3 Hz), 8.17 (2H, d, J=8.3 Hz), 10.09 (1H, s) MASS (ESI+):m/z 433.1 (M+H).

The following compound was obtained in substantially the same manner as that of Preparation 13.

Preparation 14

### 4-[5-[4-[4-(cis-4-methylcyclohexyl)-1-piperazinyl]phenyl]-1,3,4-thiadiazol-2-yl]benzaldehyde

NMR (CDCl₃, σ):0.95 (3H, d, J=6.9 Hz), 1.4-1.85 (9H, m), 2.15-2.3 (1H, m), 2.6-2.8 (4H, m), 3.3-3.4 (4H, m), 6.97 (2H, d, J=9.0 Hz), 7.8-8.15 (4H, m), 8.17 (2H, d, J=8.3 Hz), 10.09 (1H, s) MASS (ESI+):m/z 447.3 (M+H)

The following compound was obtained in substantially the same manner as that of Preparation 13.

Preparation 15

### 4′-[4-[(1s,4s)-1-methoxy-1,1′-bi(cyclohexyl)-4-yl]-1-piperazinyl]-1,1′-biphenyl-4-carbaldehyde

NMR (CDCl₃, σ):0.8-1.9 (19H, m), 2.15-2.35 (1H, m), 2.7-2.85 (4H, m), 3.11 (3H, s), 3.2-3.35 (4H, m), 7.00. (2H, d, J=8.9 Hz), 7.57 (2H, d, J=8.9 Hz), 7.72 (2H, d, J=8.3 Hz), 7.91 (2H, d, J=8.3 Hz), 10.01 (1H, s) MASS (ESI+):m/z 460.65 (M+H)

The following compound was obtained in substantially the same manner as that of Preparation 13.

Preparation 16

### 4-[5-[4-(4-cyclohexyl-4-methoxy-1-piperidyl)phenyl]-1,3,4-thiadiazol-2-yl]benzaldehyde

NMR (CDCl₃, σ):0.75-1.9 (15H, m), 3.0-3.25 (5H, m), 3.55-3.85 (2H, m), 6.97 (2H, d, J=8.9 Hz), 7.89 (2H, d, J=8.9 Hz), 8.00 (2H, d, J=8.3 Hz), 8.17 (2H, d, J=8.3 Hz), 10.08 (1H, s) MASS (ESI+):m/z 462.3 (M+H)

Preparation 17

A mixture of 4-[5-[4-(4-cyclohexyl-1-piperazinyl)phenyl]-1,3,4-thiadiazol-2-yl]benzoic acid (3 g), N,O-dimethylhydroxylamine hydrochloride (718 mg), 1-hydroxybenzotriazole (904 mg), 1-ethyl-3-(3′-dimethylaminopropyl)carbodiimide hydrochloride(1.28 g) and diisopropylethylamine (1.4 ml) in N,N-dimethylformamide (60 ml) was stirred for 1.5 hour. And the reaction mixture was poured into water. The resulting precipitates were filtered, washed with water and acetonitrile and dried. The residue was purified by silica gel chromatography (50:1 dichloromethane-methanol elution) to give 4-[5-[4-(4-cyclohexyl-1-piperazinyl)phenyl]-1,3,4-thiadiazol-2-yl]-N-methoxy-N-methylbenzamide (2.2 g).

NMR (DMSO-d₆+D₂O, σ):1.05-2.0 (10H, m), 2.2-2.4 (1H, m), 2.74 (4H, t, J=5.0 Hz), 3.34 (4H, t, J=5.0 Hz), 3.40 (3H, s), 3.57 (3H, s), 6.96 (2H, d, J=8.9 Hz), 7.81 (2H, d, J=8.4 Hz), 7.90 (2H, d, J=8.9 Hz), 8.04 (2H, d, J=8.4 Hz) MASS (ESI+):m/z 492.3 (M+H)

Preparation 18

A mixture of ethyl 4-(4-butyl-4-methoxy-1-piperidyl)benzoate (2.87 g) and 10% sodium hydroxide solution (14.4 ml) in a mixed solvent of methanol (28.7 ml) and tetrahydrofuran (57.4 ml) was refluxed for 2 hours. After cooling to ambient temperature, the reaction mixture was poured into cold water, and the mixture was adjusted to pH 3 with 1.0 mol/l hydrochloric acid. The resulting precipitates were filtered, washed with water and then dried to give 4-(4-butyl-4-methoxy-1-piperidyl)benzoic acid (2.41 g).

NMR (DMSO-d₆, σ):0.88 (3H, t, J=6.6 Hz), 1.1-1.6 (8H, m), 1.65-1.85 (2H, m), 2.9-3.15 (2H, m), 3.08 (3H, s), 3.45-3.65 (2H, m), 6.94 (2H, d, J=8.9 Hz), 7.74 (2H, d, J=8.9 Hz), 12.20 (1H, brs), MASS (m/z): 290.4 (M-1)

Preparation 19

A mixture of ethyl 4-(2-aminoacetyl)benzoate hydrochloride (2 g), 4-(4-butyl-4-methoxy-1-piperidyl)benzoic acid(2.39 g), 1-hydroxybenzotriazole (1.22 g), 1-ethyl-3-(3′-dimethylaminopropyl)carbodiimide hydrochloride (1.73 g) and triethylamine (1.26 ml) in dichloromethane (40 ml) was stirred for 20 hours at room temperature. And the reaction mixture was poured into water. The resulting precipitates were filtered and dried. The residue was purified by silica gel chromatography (5:1-1:1 dichloromethane-ethylacetate elution) to give ethyl 4-[2-[4-(4-butyl-4-methoxy-1-piperidyl)benzoylamino]acetyl]-benzoate (1.4 g).

NMR (CDCl₃, σ):0.92 (3H, t, J=6.4 Hz), 1.1-1.7 (11H, m), 1.75-1.95 (2H, m), 3.05-3.3 (2H, m), 3.18 (3H, s), 3.45-3.65 (2H, m), 4.43 (2H, q, J=7.1 Hz), 4.98 (2H, d, J=4.1 Hz), 6.89 (2H, d, J=7.8 Hz), 7.0-7.15 (1H, m), 7.78 (2H, d, J=7.8 Hz), 8.09 (2H, d, J=8.4 Hz), 8.19 (2H, d, J=8.4 Hz), MASS (m/z): 503.2 (M+23)

Preparation 20

A mixture of ethyl 4-[2-[4-(4-butyl-4-methoxy-1-piperidyl)benzoylamino]acetyl]benzoate (1.4 g) and diphosphorus pentasulfide (971 mg) in dimethoxyethane (42 ml) was refluxed for 1.5 hour. To the reaction mixture was added triethylamine (0.812 ml). The mixture was refluxed for 1.5 hour. To the reaction mixture was added water. The precipitate was collected by filtration, washed with water and dried under reduced pressure to give ethyl 4-[2-[4-(4-butyl-4-methoxy-1-piperidyl)phenyl]-1,3-thiazol-5-yl]benzoate (816 mg).

NMR (CDCl₃, σ):0.92 (3H, t, J=6.3 Hz), 1.2-2.0 (13H, m), 3.0-3.3 (2H, m), 3.19 (3H, s), 3.45-3.6 (2H, m), 4.40 (2H, q, J=7.1 Hz), 6.96 (2H, d, J=8.9 Hz), 7.64 (2H, d, J=6.7 Hz), 7.85 (2H, d, J=8.9 Hz), 8.0-8.15 (3H, m), MASS (m/z): 579.2 (M+1)

Preparation 21

To a solution of ethyl 4-[2-[4-(4-butyl-4-methoxy-1-piperidyl)phenyl]-1,3-thiazol-5-yl]benzoate (0.81 g) in tetrahydrofuran (24.3 ml) was added lithium aluminum hydride (96.3 mg) and stirred for 30 minutes at room temperature. To the reaction mixture was added ethylacetate (0.992 ml) and stirred for 3 hours at room temperature. To the reaction mixture was added water. And the reaction mixture was adjusted to pH 2.5 with 1N HCl and the resulting precipitate was collected by filtration, washed with water and acetonitrile, and dried. The residue was purified by silica gel chromatography (100:1-50:1 dichloromethane-methanol elution) to give 4-[2-[4-(4-butyl-4-methoxy-1-piperidyl)phenyl]-1,3-thiazol-5-yl]phenylmethanol (578.9mg).

NMR (CDCl₃, σ):0.93 (3H, t, J=6.5 Hz), 1.2-2.0 (11H, m), 3.05-3.3 (2H, m), 3.19 (3H, s), 3.4-3.6 (2H, m), 4.73 (2H, d, J=4.7 Hz), 6.95 (2H, d, J=8.9 Hz), 7.40 (2H, d, J=8.3 Hz), 7.58 (2H, d, J=8.3 Hz), 7.83 (2H, d, J=8.9 Hz), 7.93 (1H, s), MASS (m/z): 437.2 (M+l)

Preparation 22

A mixture of 4-[2-[4-(4-butyl-4-methoxy-1-piperidyl)phenyl]-1,3-thiazol-5-yl]phenylmethanol (570 mg) and minganese dioxide (2.27 g) in chloroform (57 ml) was stirred for 8 hours at ambient temperature. The mixture was filtered by celite and the filtrate was concentrated under reduced pressure to give 4-[2-[4-(4-butyl-4-methoxy-1-piperidyl)phenyl]-1,3-thiazol-5-yl]benzaldehyde (522.7 mg)

NMR (CDCl₃, σ):0.93 (3H, t, J=6.4 Hz), 1.2-1.7 (8H, m), 1.8-2.0 (2H, m), 3.05-3.25 (2H, m), 3.19 (3H, s), 3.45-3.65 (2H, m), 6.95 (2H, d, J=8.9 Hz), 7.74 (2H, d, J=8.3 Hz), 7.85 (2H, d, J=8.9 Hz), 7.91 (2H, d, J=8.3 Hz), 8.09 (1H, s), 10.01 (1H, s), MASS (m/z) : 435.2 (M+l)

The Starting Compounds used and the Object Compounds obtained in the following Examples 1 to 6 are given in the table as below, in which the formulas of the starting compounds are in the upper column, and the formulas of the object compounds are in the lower column, respectively.

Abbreviations used herein have the following meanings:

| ABBREVIATION | DEFINITION |
|---|---|
| Me | methyl |
| Fmoc | fluorenylmethoxycarbonyl |
| | to be continued on the next page |

| Example | |
|---|---|
| No. | Formula |
| 1 | 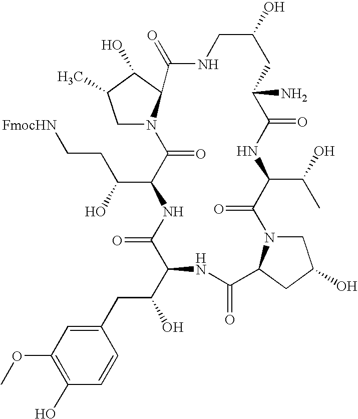 |
| | 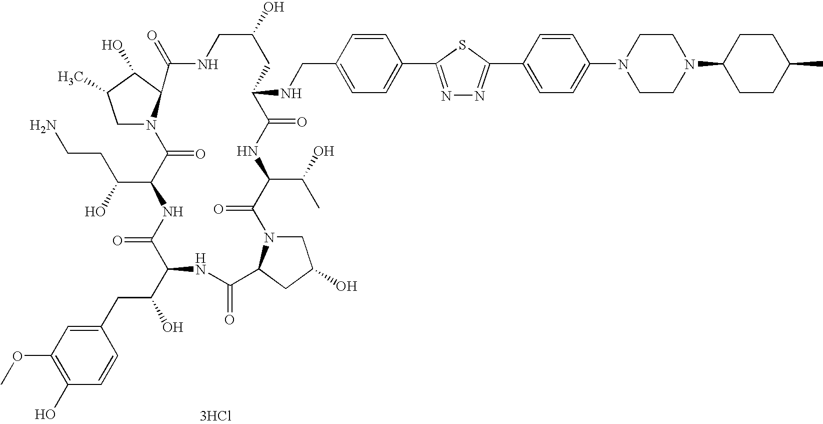 |
| 2 | 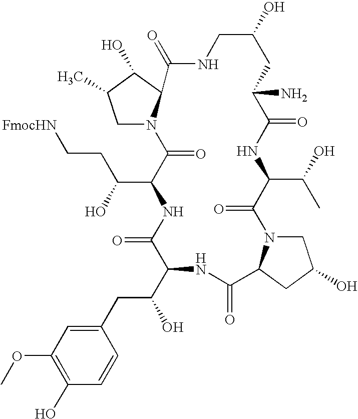 |
| | 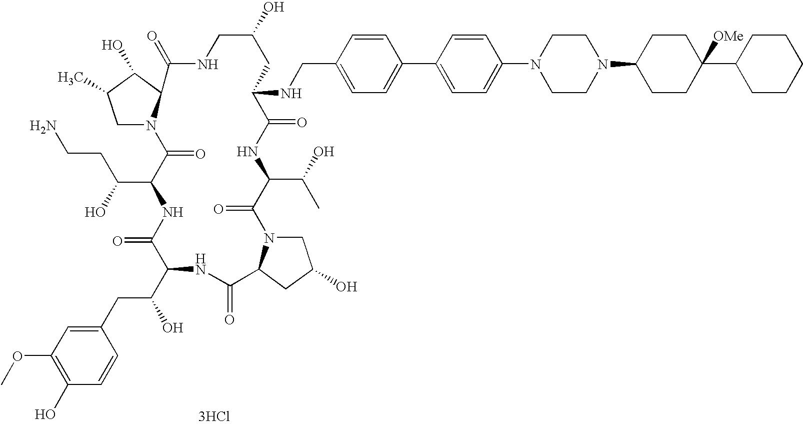 |
| 3 | 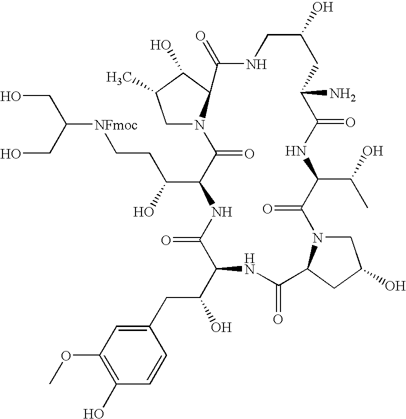 |
| | 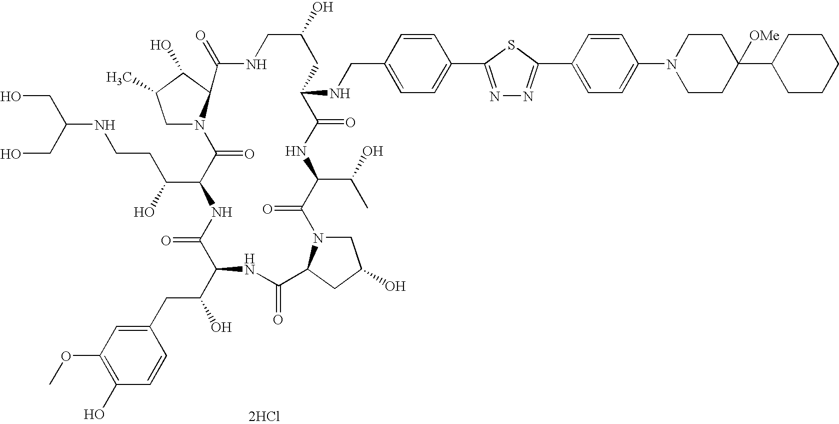 |
| 4 | 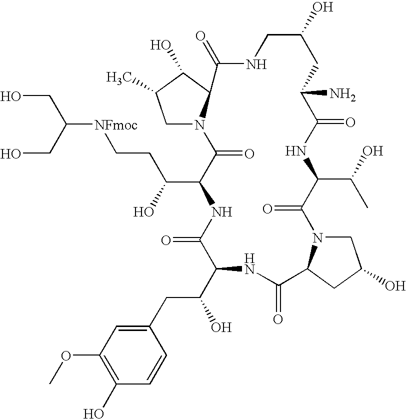 |
| | 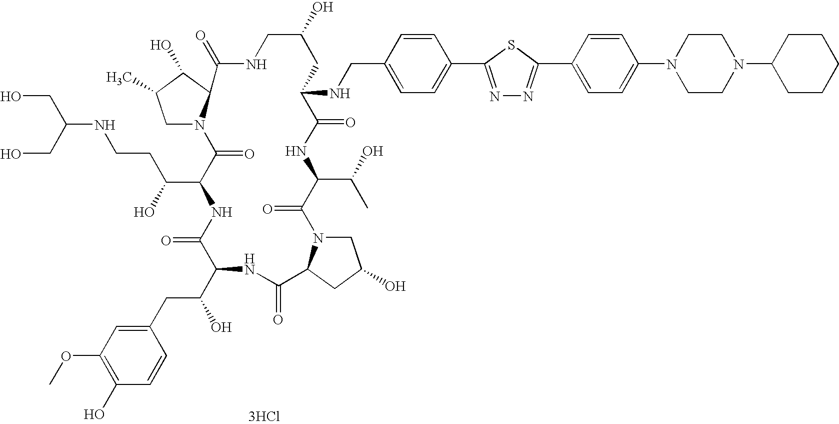 |
| 5 | 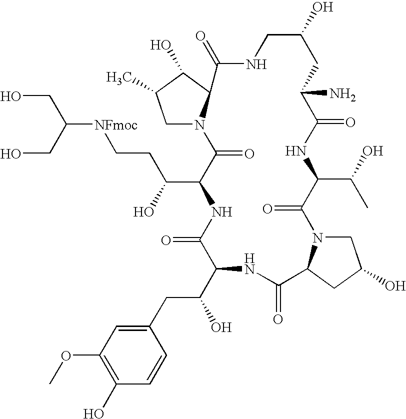 |
| | 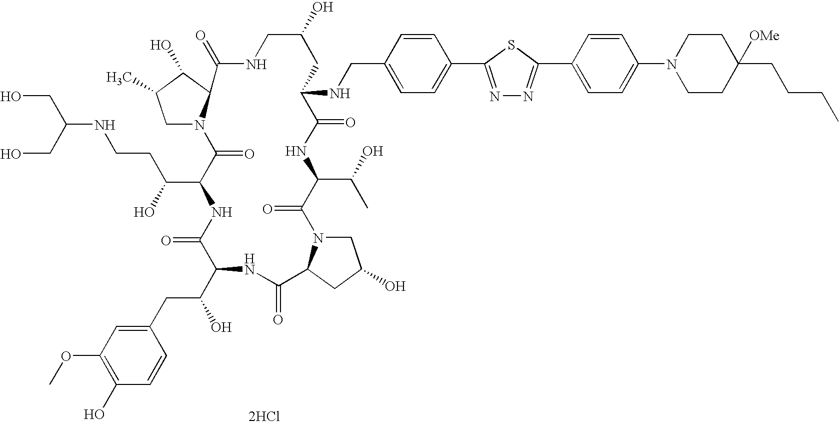 |
| 6 | 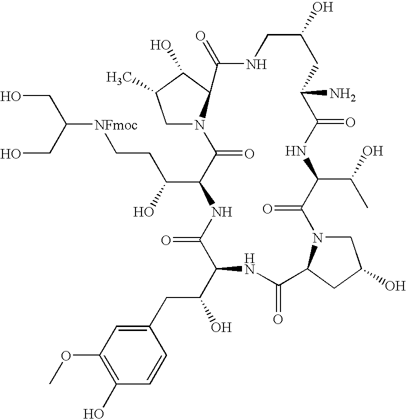 |
| | 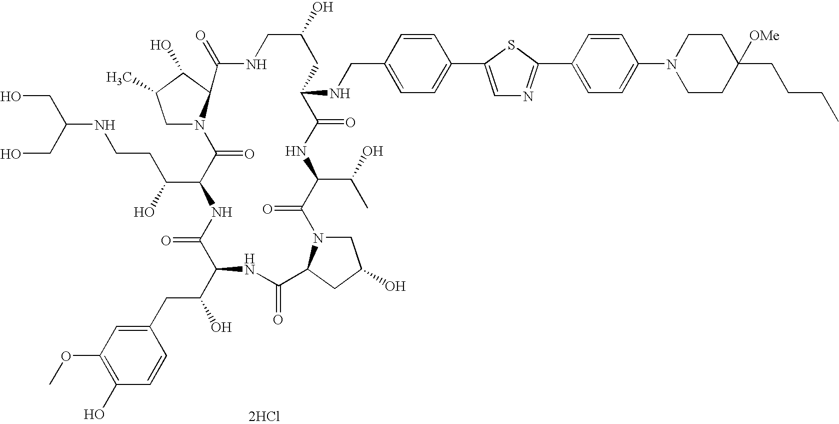 |

### EXAMPLE 1

A mixture of starting compound (1) (250 mg), 4-[5-[4-[4-(cis-4-methylcyclohexyl)-1-piperazinyl]phenyl]-1,3,4-thiadiazol-2-yl]benzaldehyde (160 mg), sodium cyanoborohydride (30 mg), and acetic acid (41 μl) in N,N-dimethylformamide (2.5 ml), methanol (2.5 ml) and dichloromethane (3.75 ml) was stirred for 1 day at ambient temperature. The reaction mixture was added piperidine (0.236 ml), and stirred for 2 hours at ambient temperature. The solution was evaporated under reduced pressure to remove dichloromethane, then added ethyl acetate. The resulting precipitates were collected by filtration and dried in vacuo. The precipitates were purified by column chromatography on ODS. The fractions containing the object compound were combined, and evaporated under reduced pressure to remove acetonitrile. And the residue was adjusted to pH 3 with 1N HCl, and was lyophilized to give object compound (1) (201 mg).

NMR (DMSO-d₆+D₂O, δ): 0.85-1.1 (6H, m), 1.19 (3H, d, J=6.1 Hz), 1.45-4.9 (52H, m), 6.5-6.75 (3H, m), 7.19 (2H, d, J=8.8 Hz), 7.70 (2H, d, J=8.2 Hz), 7.93 (2H, d, J=8.8 Hz), 8.07 (2H, d, J=8.2 Hz) MASS (ESI+) m/z: 628.07 ((M/2)+H)

The following object compounds [Example 2 to 6] were obtained according to a similar manner to that of Example 1.

### EXAMPLE 2

NMR (DMSO-d₆+D₂O, δ): 0.8-4.5 (63H, m), 0.96 (3H, d, J=6.5 Hz), 1.17 (3H, d, J=5.9 Hz), 4.6-4.85 (2H, m), 6.5-6.75 (3H, m), 7.11 (2H, d, J=8.9 Hz), 7.54 (2H, d, J=8.2 Hz), 7.64 (2H, d, J=8.9 Hz), 7.70 (2H, d, J=8.2 Hz) MASS (ESI+) m/z: 1291.6 (M+H)

### EXAMPLE 3

NMR (DMSO-d₆+D₂O, δ): 0.8-1.35 (12H, m), 1.5-4.5 (53H, m), 4.6-4.85 (2H, m), 6.5-6.75 (3H, m), 7.14 (2H, d, J=8.9 Hz), 7.68 (2H, d, J=8.3 Hz), 7.86 (2H, d, J=8.9 Hz), 8.06 (2H, d, J=8.3 Hz) MASS (ESI+) m/z: 672.47 ((M/2)+H)

### EXAMPLE 4

NMR (DMSO-d₆+D₂O, δ): 0.95 (3H, d, J=6.7 Hz), 1.0-4.55 (59H, m), 4.65-4.85 (2H, m), 6.5-6.75 (3H, m), 7.19 (2H, d, J=8.7 Hz), 7.69 (2H, d, J=8.2 Hz), 7.92 (2H, d, J=8.7 Hz), 8.07 (2H, d, J=8.2 Hz) MASS (ESI+) m/z: 1337.2 (M+Na)

### EXAMPLE 5

NMR (DMSO-d₆+D₂O, δ): 0.89 (3H, t, J=6.6 Hz), 0.95 (3H, d, J=6.8 Hz), 1.05-4.5 (57H, m), 4.6-4.85 (2H, m), 6.5-6.75 (3H, m), 7.08 (2H, d, J=8.8 Hz), 7.68 (2H, d, J=8.2 Hz), 7.83 (2H, d, J=8.8 Hz), 8.06 (2H, d, J=8.2 Hz) MASS (ESI+) m/z: 1340.4 (M+Na)

### EXAMPLE 6

NMR (DMSO-d₆+D₂O, δ):0.8-4.5 (63H, m), 4.6-4.9 (2H, m), 6.5-6.8 (3H, m), 7.09 (2H, d, J=8.9 Hz), 7.56 (2H, d, J=8.3 Hz), 7.65-7.9 (4H, m), 7.26 (1H, s), MASS (m/z):MS (m/z): 1340.4 (M+23)

1. A lipopeptide compound of the following general formula (I): 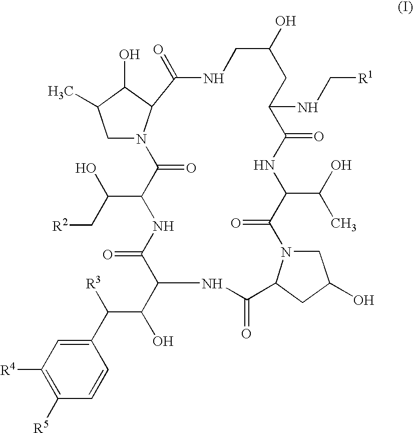
wherein
R¹ is aryl substituted with one or more suitable substituent(s),
R² is carbamoyl or amino(lower)alkyl which may be substituted with lower alkyl substituted with one or more hydroxy,
R³ is hydrogen or hydroxy,
R⁴ is hydrogen, hydroxy, lower alkoxy or amino(lower)alkoxy, and
R⁵ is hydroxy or acyloxy,
or a salt thereof:

2. A compound of claim 1, wherein
R¹ is (1) aryl substituted with heterocyclic group which may be substituted with aryl which may be substituted with optionally substituted heterocyclic group or
(2) aryl substituted with aryl which may be substituted with heterocyclic group which may be substituted with optionally substituted cyclo(lower)alkyl.

3. A compound of claim 2, wherein
R¹ is (1) phenyl substituted with heterocyclic group selected form the group consisting of thiadiazolyl, thiazolyl, piperazinyl and piperidyl, each of which may be substituted with phenyl which may be substituted with heterocyclic group selected from the group consisting of thiadiazolyl, thiazolyl, piperazinyl and piperidyl, each of which may be substituted with one or two substituent(s) selected from the group consisting of optionally substituted with cyclo(lower)alkyl, lower alkoxy and lower alkyl or
(2) phenyl substituted with phenyl which may be substituted with heterocyclic group selected from the group consisting of thiadiazolyl, thiazolyl, piperazinyl and piperidyl, each of which may be substituted with cyclo(lower)alkyl which may be substituted with one or two substituent(s) selected from the group consisting of optionally substituted cyclo(lower)alkyland lower alkyl.

4. A compound of claim 3, wherein
R¹ is (1) phenyl substituted with thiadiazolyl substituted with phenyl substituted with piperazinyl substituted with cyclo(lower)alkyl which may be substituted with one or two lower alkyl,
(2) phenyl substituted with thiadiazolyl substituted with phenyl substituted with piperidyl substituted with one or two substituent(s) selected from the group consisting of cyclo(lower)alkyl, lower alkyl and lower alkoxy,
(3) phenyl substituted with phenyl substituted with piperazinyl substituted with cyclo(lower)alkyl substituted with cyclo(lower)alkyl and lower alkoxy or
(4) phenyl substituted with thiazolyl substituted with phenyl substituted with piperidyl substituted with one or two substituent(s) selected from the group consisting of cyclo(lower)alkyl, lower alkyl and lower alkoxy,
R² is carbamoyl or amino(lower)alkyl which may be substituted with lower alkyl substituted with two hydroxy,
R³ is hydrogen,
R⁴ is hydrogen, hydroxy, lower alkoxy or amino(lower)alkoxy, and
R⁵ is hydroxy.

5. A compound of claim 4, wherein
R¹ is (1) phenyl substituted with thiadiazolyl substituted with phenyl substituted with piperazinyl substituted with cyclohexyl which may be substituted with methyl,
(2) phenyl substituted with thiadiazolyl substituted with phenyl substituted with piperidyl substituted with cyclohexyl and methoxy,
(3) phenyl substituted with thiadiazolyl substituted with phenyl substituted with piperidyl substituted with butyl and methoxy,
(4) phenyl substituted with phenyl substituted with piperazinyl substituted with cyclohexyl substituted with methoxy and cyclohexyl or
(5) phenyl substituted with thiazolyl substituted with phenyl substituted with piperidyl substituted with butyl and methoxy,
R² is amino (lower) alkyl which may be substituted with lower alkyl substituted with two hydroxy,
R³ is hydrogen,
R⁴ is lower alkoxy, and
R⁵ is hydroxy.

6. A process for preparing a lipopeptide compound (I) of claim 1, or a salt thereof, which comprises,
1) subjecting a compound (II) of the formula: 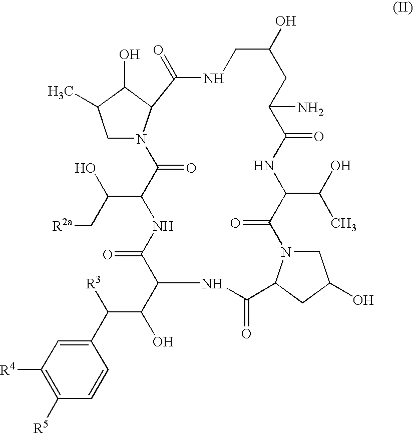
wherein R³, R⁴ and R⁵ are defined in claim 1, and
R^{2a} is protected amino(lower)alkyl which may be substituted with lower alkyl substituted with one or more hydroxy,
or a salt thereof, with a compound (V) of the formula:
R¹—CHO (V)
wherein R¹ is defined in claim 1,
or a salt thereof to the condensing reaction, and then to the elimination reaction, to give a compound (Ia) of the formula: 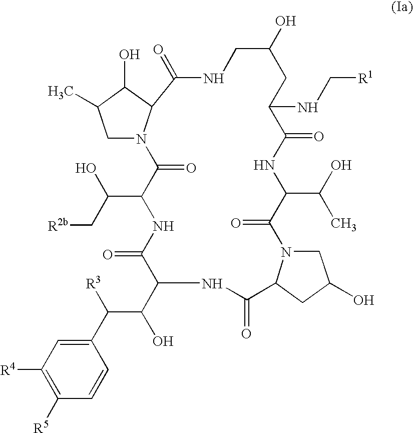
wherein R¹, R³, R⁴ and R⁵ are defined above,
R²b is amino(lower)alkyl which may be substituted with lower alkyl substituted with one or more hydroxy or a salt thereof, or
ii) subjecting a compound (III) of the formula: 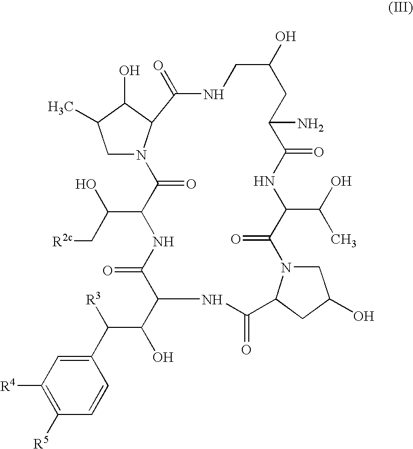
wherein R³, R⁴ and R⁵ are defined in claim 1, and
R^{2c} is carbamoyl,
or a salt thereof, with a compound (V) of the formula:
R¹—CHO (V)
wherein R¹ is defined in claim 1, or a salt thereof to the condensing reaction, to give a compound (Ib) of the formula: 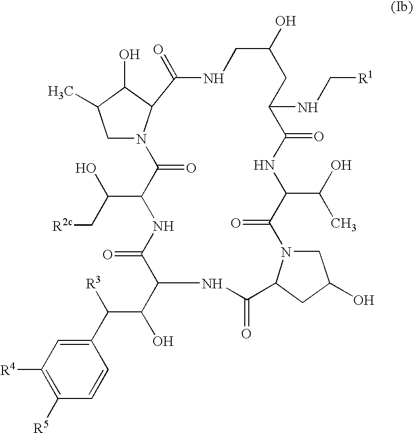
wherein R¹, R^{2c}, R³, R⁴ and R⁵ are defined above, or a salt thereof.

7. A pharmaceutical composition which comprises, as an active ingredient, a compound of claim 1 or a pharmaceutically acceptable salt thereof in admixture with pharmaceutically acceptable carriers or excipients.

8. Use of a compound of claim 1 or a pharmaceutically acceptable salt thereof for the manufacture of a medicament.

9. A compound of claim 1 or a pharmaceutically acceptable salt thereof for use as a medicament.

10. A method for the prophylactic and/or therapeutic treatment of infectious diseases caused by pathogenic microorganisms, which comprises administering a compound of claim 1 or a pharmaceutically acceptable salt thereof to a human being or an animal.

11. A commercial package comprising the pharmaceutical composition of claim 7 and a written matter associated therewith, wherein the written matter states that the pharmaceutical composition can or should be used for preventing or treating infections disease.

12. An article of manufacture, comprising packaging material and the compound (I) identified in claim 1 contained within said packaging material, wherein said the compound (I) is therapeutically effective for preventing or treating infectious diseases, and wherein said packaging material comprises a label or a written material which indicates that said compound (I) can or should be used for preventing or treating infectious diseases.
